(19) United States
(12) Patent Application Publication
(10) Pub. No.: US 2010/0108075 A1
(43) Pub. Date: May 6, 2010

(54) Tracheal or Tracheostomy Tubular Arrangement

(75) Inventor: Armin Singvogel, Remsbeck (DE)

Correspondence Address: BAKER & HOSTETLER LLP, WASHINGTON SQUARE, SUITE 1100, 1050 CONNECTICUT AVE. N.W., WASHINGTON, DC, 20036-5304, US

(73) Assignee: Willy Rusch GmbH, kernen-Rommelshausen (DE)

(21) Appl. No.: 12/529,677

(22) PCT Filed: Mar. 13, 2008
(86) PCT No.: PCT/EP08/02032

§ 371 Date: Sep. 2, 2009

(30) Foreign Application Priority Data
Mar. 13, 2007 (DE) 10 2007 011 930

(51) Int. Cl. A61M 16/04

(52) U.S. Cl. 128/207.14

(57) ABSTRACT

The present invention provides a tracheal or tracheostomy tubular arrangement 1 having an outer tube 2 and an inner cannula 3, which can be inserted into the outer tube 2 and pulled out of the same, wherein the inner cannula 3 has at least one plastic layer 21 forming at least the inner surface 31 of the same, and a support structure supporting the plastic layer 21. For this purpose, the support structure is configured by a tubular interlacing 20 made of threads and/or fibers.

### CROSS-REFERENCE TO RELATED APPLICATIONS

This application is a national stage of International Application No. PCT/EP2008/002032, filed Mar. 13, 2008, which claims priority to foreign German Application No. DE 10 2007 011 930, filed Mar. 13, 2007, the disclosure of each application is hereby incorporated by reference in their entirety.

### FIELD OF THE INVENTION

The invention relates to a tracheal or tracheostomy tube configuration with an external tube and an internal cannula which is insertable into the external tube and extractable from it.

### BACKGROUND OF THE INVENTION

Tracheal or tracheostomy tube configurations are used, among other things, in the artificial respiration of patients and in intensive medicine. When so used, a tracheostomy tube configuration is inserted into the airway through a cut in the airway while a tracheal tube configuration is inserted through the mouth or through the nose of the patient into the airway. Tracheal or tracheostomy tube configurations are distinguished essentially merely by their machine-side end areas which are attachable to a respirator since these end areas are to be affixed to various parts of the body.

Generic tracheal or tracheostomy tube configurations are, for example, known from WO 91/12844 A1. The external tube (outer tube) of a tracheal or tracheostomy tube configuration of this type is provided for insertion into the airway. The internal cannula is inserted through the external tube and can be removed and replaced at periodic intervals, namely when so much secretion has collected in the cannula that replacement is considered appropriate.

The insertion of the internal cannula into the tube can, however, be difficult. In order to achieve as large an internal diameter of the internal cannula as possible, and thus as little resistance to respiration as possible, the internal cannula should have as thin a wall as possible and the external diameter of the internal cannula should correspond essentially to the internal diameter of the tube. Due to the relationships of the diameters of the internal cannula to the tube the internal cannula on insertion usually contacts the internal wall of the tube over a large surface area. From this a great frictional resistance follows on insertion.

Due to this frictional resistance and the thin walls and flexibility of the internal cannula, kinking of the internal cannula frequently occurs during insertion. To solve this problem WO 91/12844 A1 proposes to dispose, around the external surface of the internal cannula, a filament, e.g. of metal in the form of a spiral. With an external spiral of this type the radial rigidity of the internal cannula is increased and the coefficient of friction between the internal wall of the tube and the external surface of the internal cannula is reduced.

In order to achieve sufficient rigidity the material strength of the external spiral must, however, be relatively great so that the internal diameter of the internal cannula decreases correspondingly with increasing resistance to respiration.

### SUMMARY OF THE INVENTION

The invention is based on the objective of providing a tracheal or tracheostomy tube configuration and a process for producing the internal cannula of the tracheal or tracheostomy tube configuration which avoid the disadvantages of the state of the art, where in particular the stability with respect to kinking in the case of thin-walled internal cannulas will be improved.

A tracheal or tracheostomy tube configuration according to the invention comprises an external tube and an internal cannula which is insertable into the tube and extractable from it. Along with this the internal cannula comprises at least one plastic layer implementing at least its internal surface and a support structure supporting the plastic layer. According to the invention the support structure is implemented by a tubular braid made of thread and/or fibers.

The plastic layer implements, together with the tubular braid, a closed tube. Due to the fact that the internal surface is implemented by the plastic layer, it is relatively smooth so that respiratory secretions can adhere and not be removed by turbulences in the respiratory air flow which form at rough points. Since a braid comprises many threads interwoven with one another, the threads can be implemented so as to be much thinner than in the case of a merely helical support structure. The internal cannula can thus be implemented so as to have very thin walls. Furthermore, a braid has the advantage that there is a stabilization in several directions. The braid can, for example, comprise a plurality of threads interwoven with one another and crossing one another in a helical pattern in such a way that in the support structure there are openings between the threads, where said openings have the shape of a diamond and are separated from one another by the threads. The plastic layer therefore only has to span these diamond-shaped openings, whereas on the contrary with a support structure comprising merely a filament disposed in the form of a helix a continuous area over the entire length of the internal cannula has to be spanned. The plastic layer can therefore be implemented so as to have uniform rigidity and be substantially thinner. The internal cannula implemented according to the invention is thus, with a very slight wall thickness, flexible and yet stable with respect to kinking.

Preferably the support structure is essentially enveloped completely by the plastic layer in such a way that the external surface of the internal cannula is implemented by the plastic layer. An internal cannula of this type can be produced in a simple way, for example, by introducing a support structure in plastic in a manner customary for tubes.

Particularly preferably the internal cannula's internal surface which is implemented by the plastic layer is essentially smooth and the external surface implemented by the plastic layer models the external structure of the braid. The implemented external structure of the braid implements a surface which is rough relative to the internal surface of the internal cannula. On insertion of the internal cannula into the tube a rough surface of this type has less contact with the internal surface of the tube so that the friction between these surfaces is less than with smooth surfaces.

If the threads or fibers of the braid comprise plastic and not, for example, metal, which would also be possible, a particularly low weight with great stability of the internal cannula is achievable. The threads or fibers implementing the braid can, for example, comprise polyester, Trevira, polyamide, polyethylene, polyimide, polyethersulfone, copolyamide, or fluorocarbon. Threads or fibers of different materials can also be interwoven with one another. Preferably, however, a polyester braid is used. The thread thickness of the plastic threads can, for example, be 0.05 to 0.3 mm. Furthermore, in a plastic braid it is possible to easily introduce additional functions, such as tubular openings to enable speech, in an existing tubular insert, i.e. when the tube is applied in the airway of a patient.

The threads or fibers of the braid are advantageously open or connected, at least in the area of the free ends of the internal cannula, and are treated with heat as an entire part in order to prevent a curling of the ends.

Likewise welding, laminating, or gluing are possible.

The ends can also be protected by a thin film which is generated by a so-called thin, flexible shrunk-on tube.

An additional possibility for protecting the end threads from curling up is to use a form-locking part similar to a clip connection with an internal ring and an external ring. This possibility can also be applied to punched-out eyes and/or holes.

Advantageously the plastic layer comprises, for example, silicone rubber or another elastomer such as TPE, PUR, or PVC. A plastic layer of this type can be applied to the support structure in a simple manner by dipping a tubular braid as a support structure in a liquid silicone rubber mass.

Furthermore, in an internal cannula of this type aids made of silicone rubber can be provided in the material composite. For example, a grip can be molded on the machine-side end area of the internal cannula.

Preferably a stop is provided as an aid at one of the free ends of the internal cannula, where the stop, for example, can be implemented by a silicone hose piece which is glued by means of a silicone adhesive, using its internal surface, onto the external surface of the internal cannula. A stop of this type enables a positioning with precise fit of the internal cannula in the external tube. In this case the stop is structured so that any additional adaptation to the external connector is not disturbed.

Advantageously, a filler which provides an X-ray shadow can be provided in the plastic layer and/or in the braid and/or at least the internal surface of the internal cannula can comprise an antimicrobially active coating, in particular a silver coating.

In a process according to the invention and for producing an internal cannula for a tracheal or tracheostomy tube configuration in which the support structure of the internal cannula is essentially completely enveloped by the plastic layer (closed internal cannula coating) in such a way that also the external surface of the internal cannula is implemented by the plastic layer, a tubular support structure made of a braid made of threads and/or fibers is provided and the plastic layer is produced by dipping the braid in a liquid plastic, preferably liquid silicone rubber, and curing the plastic layer. The closed internal cannula coating is preferably done by a merely one-time dipping, which leads to a particularly thin, closed coating. In so doing, no core rod is used. Simple dipping is essentially faster and less complicated than extrusion processes for the plastic coating of a braid. The process according to the invention is thus very economical.

Similar tubes can also be produced in the injection-molding process by laying in the braid or by spraying the braid.

With a suitable choice of the geometry of the braid of the support structure, by dipping it is possible without additional measures to achieve the result that the internal cannula's internal surface implemented by the plastic layer is essentially smooth and the external surface implemented by the plastic layer models the external structure of the braid. In such a way a smooth internal coating with a rough external coating is directly achievable, which simplifies to a very great extent the internal cannula's insertion into and extraction from the tube so that no blocking due to kinking of the internal cannula occurs. Said surface structure can arise by the internal surface having a smaller radius than the external surface. Due to the surface tension of the liquid material the still liquid plastic layer in the area of the interstices between neighboring threads or fibers of the braid is drawn radially inwards. The membrane implemented between neighboring fibers by the plastic layer thus runs in the range of the internal radius of the internal cannula, which leads to said surface structure. If the braid comprises threads running in parallel and crossing so that between neighboring thread there are diamond-shaped interstices, a thread spacing of 20 braid diamonds in the range of 13 plus/minus 5 mm has proven itself expedient when using silicone rubber. The braid diamonds have therefore a diameter in the range of ca. 1 mm.

It is obvious that it is also possible in the production of an internal cannula to use threads and braids whose cross sections are implemented so that they are flat with respect to their alignment relative to the internal surface and bent or curved with respect to their alignment relative to the outer surface.

The invention will be explained in more detail with the aid of an embodiment example with reference to the drawings.

### BRIEF DESCRIPTION OF THE DRAWINGS

FIG. 1 shows a tracheostomy tube configuration according to the invention.

FIG. 2 shows the internal cannula of the tracheostomy tube configuration according to the invention and as seen in FIG. 1.

FIG. 3 shows an enlarged cross-sectional representation of a part of the internal cannula of the tracheostomy tube configuration according to the invention and as seen in FIG. 1.

The figures of the drawings show the object according to the invention in a strongly schematized manner and are not to be understood as drawn to scale. The individual components of the object according to the invention are represented so that their design can be shown well.

### DETAILED DESCRIPTION OF THE INVENTION

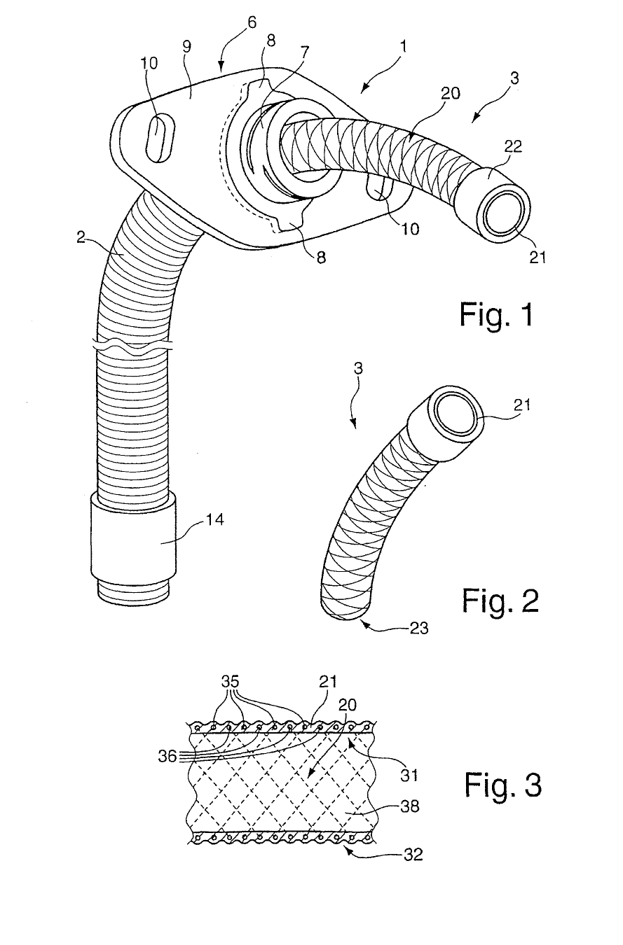

In FIG. 1 a tracheostomy tube configuration 1 according to the invention is shown. The tracheostomy tube configuration 1 comprises an external tube 2 and an internal cannula 3. The internal cannula 3 is insertable into the external tube 2 and extractable from it. In the figure the internal cannula 3 is partially inserted into the tube 2. The tube 2 comprises on its machine-side end, which is attachable to a respirator, a flange 6. On the flange 6 a screw thread 7 and fixing elements 8 are implemented, whereby a locking screwing in of a connecting element of a breathing tube system of the respirator is possible. Furthermore, a collar 9 with openings 10 for fastening the collar 9 on the neck of a patient is provided on the flange 6. Furthermore, in the area of the patient-side end of the tube 2, where said end is to be disposed in the airway of the patient, an inflatable collar 14 is disposed. This collar 14 is inflatable from outside in the manner of a balloon in order to fix the tube 2 so as to be positioned immovably at a point in the airway. The tube 2 itself is, for example, implemented by a plastic tube, where in the plastic a, for example, metal spiral is embedded.

The internal cannula 3 comprises a support structure implemented by a tubular braid 20 made of plastic threads. This support structure is molded in a plastic layer 21 which implements the internal and the external surface of the internal cannula 3. The support structure is therefore completely enveloped by the plastic layer 21 so that an airtight tube is implemented. As an aid a stop 22 is provided on the machine-side free end of the internal cannula. This stop 22 is implemented by a short silicone hose piece which is glued by means of a silicone adhesive, using its internal surface, onto the external surface of the internal cannula.

In FIG. 2 the internal cannula 3 of the tracheostomy tube configuration according to the invention and seen in FIG. 1 is shown. The patient-side end 23 of the internal cannula 3 covered by the tube in FIG. 1 is formed in no particular manner with respect to the tube, which is implemented by the support structure enveloped by the plastic layer 21. This patient-side end can be implemented simply as a cut edge of this tube.

In FIG. 3 an enlarged cross-sectional representation of a part of the internal cannula of the tracheostomy tube configuration according to the invention and seen in FIG. 1 is shown. The internal surface 31 of the internal cannula, where said internal surface is implemented by the plastic layer 21, is smooth and the external surface 32 implemented by the plastic layer 21 models the external structure of the braid 20. The external surface 32 is therefore rougher than the internal surface 31. The braid 20 is made of threads 35, 36 running in parallel and crossing over others so that between neighboring threads there are interstices 38 in the form of a diamond. That is, one part of the threads 35 runs in a left-oriented spiral around the longitudinal axis of the internal cannula and another part of the threads 36 runs in a right-oriented spiral around the longitudinal axis of the internal cannula. The threads 35, 36 running with left-orientation or right-orientation are parallel to those running with the same orientation and cross those running with the opposite orientation. In connection with this the threads 35, 36 can preferably be interwoven so that neighboring threads crossed by a thread are crossed alternately from below, i.e. on the internal wall side, and from above, i.e. on the external wall side. It is, however, possible, for example, to dispose all the threads running with a left orientation on the internal wall side and all the threads running with a right orientation on the external wall side or vice versa.

Proposed is a tracheal or tracheostomy tube configuration 1 with an external tube 2 and an internal cannula 3 which is insertable into the external tube 2 and extractable from it, where the internal cannula 3 comprises at least one plastic layer 21 implementing at least its internal surface 31 and a support structure supporting the plastic layer 21. In connection with this the support structure is implemented by a tubular braid 20 made of threads 35, 36 and/or fibers.

The invention is not restricted to the embodiment examples specified above. Rather, a number of variants are conceivable which also make use of the invention with a fundamentally different type of implementation of the features.

What is claimed is:

1. A tracheal or tracheostomy tube arrangement, comprising:
an external tube (2) and an internal cannula (3) which is insertable into the external tube (2) and extractable from it,
wherein the internal cannula (3) comprises at least one plastic layer (21) implementing at least its internal surface (31) and a support structure supporting the plastic layer (21), the support structure including a tubular braid (20) made of thread (35, 36) and/or fibers, and wherein the support structure is completely enveloped by the plastic layer (21) in such a way that the external surface (32) of the internal cannula (3) is also implemented by the plastic layer (21).

2. (canceled)

3. A tracheal or tracheostomy tube arrangement according to claim 1,
wherein the internal surface (31) of the internal cannula (3), implemented by the plastic layer (21) is essentially smooth and the external surface (32) implemented by the plastic layer (21) models the external structure of the braid (20).

4. A tracheal or tracheostomy tube arrangement according to claim 1, wherein the threads (35, 36) or fibers of the braid (20) comprise plastic.

5. A tracheal or tracheostomy tube configuration arrangement according to claim 4, wherein
the threads (35, 36) or fibers of the braid (20) are open, welded, enveloped, and/or laminated at least in the area of the free ends of the internal cannula.

6. A tracheal or tracheostomy tube arrangement according to claim 1, characterized in that wherein the plastic layer (21) comprises silicone rubber.

7. A tracheal or tracheostomy tube configuration arrangement according to claim 6, wherein
aids made of silicone rubber are provided in the material composite on the internal cannula.

8. A tracheal or tracheostomy tube arrangement according to claim 7, wherein
a stop (22) is provided as an aid on one of the free ends of the internal cannula (3), the stop (22) being implemented by a silicone hose piece, preferably glued by means of silicone adhesive onto the external surface (32) of the internal cannula (3), using its internal surface, the stop preferably being implemented in such a way that a further adaptation of attachment parts and/or aids is not obstructed by the stop.

9. A tracheal or tracheostomy tube arrangement according to claim 1, further comprising:
a filler which provides an x-ray shadow in the plastic layer (21) and in the braid (20) and that at least the internal surface (31) of the internal cannula (3) has an antimicrobially active coating, in particular a silver coating.

10. (canceled)

11. A tracheal or tracheostomy tube arrangement according to claim 1, further comprising:
a filler which provides an x-ray shadow in the braid (20).

12. A tracheal or tracheostomy tube arrangement according to claim 11, wherein at least the internal surface (31) of the internal cannula (3) has an antimicrobially active coating.

13. A tracheal or tracheostomy tube arrangement according to claim 12, wherein the antimicrobially active coating includes a silver coating.

14. A tracheal or tracheostomy tube arrangement according to claim 1, further comprising:
a filler which provides an x-ray shadow in the plastic layer (21).

15. A tracheal or tracheostomy tube arrangement according to claim 14, wherein at least the the internal surface (31) of the internal cannula (3) has an antimicrobially active coating.

16. A tracheal or tracheostomy tube arrangement according to claim 15, wherein the antimicrobially active coating includes a silver coating.

17. A method for the production of an internal cannula for a tracheal or tracheostomy tube arrangement having an external tube (2) and an internal cannula (3) which is insertable into the external tube (2) and extractable from it, wherein the internal cannula (3) comprises at least one plastic layer (21) implementing at least its internal surface (31) and a support structure supporting the plastic layer (21), the support structure including a tubular braid (20) made of thread (35, 36) and/or fibers, and wherein the support structure is completely enveloped by the plastic layer (21) in such a way that the external surface (32) of the internal cannula (3) is also implemented by the plastic layer (21), and wherein a tubular support structure made of a braid (20) made of threads (35, 36) and/or fibers is provided, and the plastic layer (21) is produced by dipping the braid (20) in a liquid plastic, preferably liquid silicone rubber, and curing the plastic layer (21).
